(19) United States
(12) Patent Application Publication
(10) Pub. No.: US 2020/0306211 A1
(43) Pub. Date: Oct. 1, 2020

(54) METHOD FOR ADAPTING DOSES OF COMBINATION THERAPIES

(71) Applicant: Pharnext, Issy-les-Moulineaux (FR)

(72) Inventors: Daniel Cohen, Saint-Cloud (FR); Ilya Chumakov, Vaux-le-Penil (FR); Mickael Guedj, Paris (FR); Rodolphe Hajj, Saint-Germain-en-Laye (FR)

(21) Appl. No.: 15/779,750

(22) PCT Filed: Nov. 30, 2015
(86) PCT No.: PCT/IB2015/002370

§ 371 Date: May 29, 2018

(51) Int. Cl. A61K 31/16; A61K 31/195; G01N 33/94

(52) U.S. Cl. CPC A61K 31/16; G01N 33/94; A61K 31/195

(57) ABSTRACT

The present invention relates to methods for improving dose finding process for combinations of several drugs and/or adapting the dosage of combinatorial treatment. More particularly the invention relates to methods for identifying more efficient dose of active pharmaceutical ingredients (APIs) within combination therapies. This invention also relates to methods for optimizing a therapeutic efficiency of combined therapies.

The present invention relates to methods for improving dose finding process for combinations of several drugs and/or adapting the dosage of combinatorial treatment. More particularly the invention relates to methods for identifying more efficient dose of active pharmaceutical ingredients (APIs) within combination therapies. This invention also relates to methods for optimizing a therapeutic efficiency of combined therapies. This invention is also of use for adapting the doses with individual specificities of subjects to be treated. The methods of the present invention are of particular interest in the course of drug development processes.

In comparison with single drug treatments, combination therapies demonstrate better efficacy, decreased toxicity and reduced development of drug resistance, and, for these advantages, are emerging as a standard for the treatment of several diseases (e.g. HIV, hypertension or cancer). Anyway, combinatorial treatments represent a promising and innovative approach in therapy for both multifactorial common diseases and rare indications of unmet medical need. However extrapolation of results and even more of doses and/or ratios for APIs from in vitro or preclinical data to humans is challenging. Strictly speaking, the known pharmacokinetics (PK) and/or pharmacodynamics (PD) data from each active ingredient developed in monotherapy cannot be directly extrapolated for combinatorial therapies considering the possible cross interference of one of the compounds on the PK/PD of the other(s) and differences in both PK and PD between animal models and human subjects. This is becoming virtually impossible when no clinical benefit is observed for the single APIs used alone, with beneficial effect that arises due to synergistic interaction between individual APIs or to a concerted action of the different APIs on various features of the disease. Thus, when moving to clinical studies in humans, the optimization of combinatorial therapies in term of dose-finding or of ratio between each API which composes the medicine has to face strong practical and ethical limitations. For these reasons, it is strongly challenging to obtain sufficient data to clearly and adequately support optimal synergy between the combined APIs in front of health agencies which could require a compelling justification for the combined use of said APIs in human rather than the use of monotherapies made of each single APIs. Indeed, a factorial design of a clinical trial can appear rapidly prohibitive as exploding exponentially as a function of the number of drugs in the combinatorial treatment and/or the number of tested doses. Also, clinical heterogeneity within patient population can give rise to subgroups of patients who don't benefit from a particular therapy as they should because of, for instance, heterogeneity of the disease or metabolic and/or physiological peculiarities which can affect said ratio(s) or doses of APIs within the combinatorial therapy.

Accordingly, there is a need for a method that could allow i) an accurate and faster dose finding/optimization for combination therapies, notably, when shifting between different phases of pre-clinical and clinical trials, or ii) to determine in vivo doses of APIs where a positive enhancing effect of combined therapy is reached, and/or iii) to adapt doses and/or ratios and or dosage form of API(s) in the combinatorial treatment in regard to specificities of a subgroup of subjects or even of a single subject.

### SUMMARY OF THE INVENTION

The present invention discloses a novel method for improving the optimal dose finding process in the course of drug development processes or for adapting/optimizing the dosage of a combinatorial treatment. By following said method, it is possible to define more effective doses for a combined therapy from preliminary data obtained in a small group of human subjects.

In a particular embodiment, the invention thus relates to a method for determining an optimal dose and/or dosage form and/or ratio for one or more active pharmaceutical ingredients (APIs) of a combinatorial treatment for a selected condition or a disease.

The invention also relates to a method for improving dose and/or dosage form and/or ratio for one or more active pharmaceutical ingredients (APIs) of a combinatorial treatment for a selected condition or a disease.

The present invention further relates to a method for, within a combinatorial treatment for a condition or a disease, improving the dose finding process of one or more active pharmaceutical ingredients (APIs) and/or a the ratio of said APIs and/or the dosage form of one or more of said APIs.

The method of the invention preferably comprises
i. selecting a condition or a disease to be treated,
ii. gathering from subjects administered with one or more doses or dosage forms of said combinatorial treatment, individual pharmacokinetics (“PK”) parameters corresponding to each API or metabolite(s) thereof,
iii. gathering from said subjects data corresponding to at least one clinical endpoint, biomarker and/or surrogate marker related to the selected condition or disease,
iv. determining, for at least one API or metabolite thereof, the clinical endpoint(s), biomarker(s) and/or surrogate marker(s) and the PK parameter(s) for which a correlation can be established,
v. optionally, selecting the most relevant clinical endpoint(s), biomarker(s) and/or surrogate marker(s) for the selected condition or disease from those determined in step iv), and
vi. determining, from the preceding step iv) or v), an optimal dose and/or dosage form and/or ratio of one or more of the APIs to be administered in said combinatorial treatment to obtain a more effective treatment of said condition or disease.

Another object of the invention resides in a method for optimizing therapeutic efficiency of a combinatorial treatment of a selected condition or a disease, said method comprising:
comparing individual PK parameters for each API of said combinatorial treatment in subjects previously treated with one or several doses or dosage forms of the APIs in the combinatorial treatment,
determining a PK data variation, and
correlating said PK data variation with an alteration of clinical outcome of the selected disease,
   said correlation allowing to determine an optimal dose or dosage form of each API for said combinatorial treatment, leading to an optimization of therapeutic efficiency.

The methods of the invention allow implementing dose finding process in a more targeted way and is thereby cost and time effective in comparison with the commonly used process of trial and error.

Moreover, when implemented during clinical stages, said methods also allow diminishing the number of tested subjects, which is particularly advantageous when compared to conventional process of dose finding during clinical trials. In this regard, the invention relates to the above mentioned method wherein determining another dose and/or ratio or dosage form of APIs of step v) comprises identifying best responder subjects to said combinatorial treatment, the PK parameters for one or more of the APIs of the combinatorial treatment in said responder subjects being indicative of a need for increasing or diminishing the dosage of one or more of the APIs, or even to modify the dosage form of one or more of said APIs, in order to obtain a more effective treatment, characterized by a particular ratio of the APIs or a particular doses of said APIs.

Said methods can be used for combinatorial treatments comprising 2, 3, 4, 5 or more APIs.

Also, said methods can be used to customize the combinatorial treatment to the particular need of a subject or of a particular group of subjects. Indeed some subjects can present some specificities (metabolic particularities, interfering treatment etc . . . ) that alter their actual impregnation with one, several, if not all the APIs of the combination therapy. Such an alteration may result in a less efficient therapy or even in an ineffective treatment or in unacceptable side effects. Consequently the invention also relates to the above mentioned method wherein determining another dose and/or ratio or dosage form of APIs according to step v) comprises comparing the selected PK parameters of an individual with the corresponding mean or median observed in a group of responder subjects to said treatment and wherein a difference is indicative of a need in increasing or diminishing the dosage of one or more of the APIs within said combinatorial treatment, to achieve the maximal therapeutic effect and/or minimize undesirable side effects of the drugs when combined.

The methods of this invention can also be used to demonstrate in vivo the superiority of the combinatorial therapy over single API medication, i.e. a positive enhancing effect of combined action of the APIs, even a synergistic activity of said combination on one or more symptoms of the disease. The invention thus also relates to the above mentioned method wherein determining the optimal dose and/or ratio of APIs of step v) comprises building from step iii) or iv) an exposure-effect relationship and determining from this relationship the doses and/or ratios for which a positive enhancing effect of combined action is reached in the subjects.

The method of the invention is of particular interest in the context of multifactorial complex diseases or conditions or of diseases for which monotherapies are not efficient while combinatorial therapies having proved to be a promising strategy [1,2]. Such diseases or conditions being, for example, memory performances, neurodegenerative disorders as Alzheimer's disease, Amyotrophic Lateral Sclerosis (ALS) and Parkinson's disease, or diabetes, or peripheral neuropathies like, for example, Charcot-Marie-Tooth disease.

In this regard, the invention also relates to a method for treating a subject having a disease with a combinatorial treatment of active pharmaceutical ingredients (APIs), said method comprising (i) determining an optimal dose or dosage form or ratio of each API for said combinatorial treatment of said disease as defined above and (ii) treating the subject with said optimal dose or dosage form or ratio of each API.

This invention can be applied on the data obtained from any mammals, and is of particular interest when related to data obtained from humans.

### DESCRIPTION OF THE FIGURES

FIG. 1: Positive correlation between the plasmatic concentrations of acamprosate measured 1.5 hour after administration of the baclofen-acamprosate mix and the Event Related Potentials (ERP) data of subjects administered with baclofen-acamprosate combination. The subjects who display the best improvement of their neuro physiological data (ERP composite score) are those for which the higher plasmatic concentrations of acamprosate are reported (r: Pearson's correlation coefficient, p<0.05: PK parameter and endpoint are significantly correlated).

FIG. 2: Positive correlation between the plasmatic concentrations of baclofen measured 1.5 hour after administration of the baclofen-acamprosate mix and the Event Related Potentials data of subjects administered with baclofen-acamprosate combination. The data clearly show that the subjects who display the best improvement of their neuro physiological data (ERP composite score) are those for which the higher plasmatic concentrations of baclofen are reported (r: Pearson's correlation coefficient, p<0.05: PK parameter and endpoint are significantly correlated).

FIG. 3: Three dimensional representation of ERP composite score of subjects administered with baclofen and acamprosate combination as a function of the AUC (Area Under Curve) determined for each of baclofen and acamprosate in these subjects (arbitrary units). The plotted surface delineates all the ratios of AUC for the drug dosages tested in the study. Peaks on the surface correspond to concentration ratios for which best cognitive performances are noticed and/or expected (subject with a highest ERP composite being the best responders), whereas valleys correspond to drug exposures for which the combinatorial treatment is less efficient in regard to ERP. Four regions (*) stand out clearly; they correspond to specific ranges of ratios between baclofen and acamprosate for which the best efficacy of the combinatorial treatment can be expected.

### DETAILED DESCRIPTION OF THE INVENTION

The present invention relates to methods for improving the efficiency of combinatorial therapies. More particularly, the invention relates to methods for selecting or improving or optimizing the dose or dosage form or ratio of one or more active pharmaceutical ingredients (APIs) of a combinatorial treatment for a condition or a disease. The methods of the invention generally comprise the steps of:
i. selecting a condition or a disease to be treated,
ii. gathering from subjects administered with one or more doses or dosage forms of said combinatorial treatment, individual pharmacokinetics (“PK”) parameters corresponding to each API or metabolite(s) thereof,
iii. gathering from said subjects data corresponding to at least one clinical endpoint, biomarker and/or surrogate marker related to the selected condition or disease,
iv. determining, for at least one API or metabolite thereof, the clinical endpoint(s), biomarker(s) and/or surrogate marker(s) and the PK parameter(s) for which a correlation can be established,
v. optionally, selecting the most relevant clinical endpoint(s), biomarker(s) and/or surrogate marker(s) for the selected condition or disease from those determined in step iv), and
vi. determining, from the preceding step iv) or v), an optimal dose and/or dosage form and/or ratio of one or more of the APIs to be administered in said combinatorial treatment to obtain a more effective treatment of said condition or disease.

The use of combinatorial therapies, though emerging as the breakthrough strategy for treating multifactorial complex diseases, raises several practical and ethical issues. Indeed they require multiple arm clinical trials resulting in several hurdles in terms of cost, study duration, logistical and ethical concerns. Moreover, one cannot rely on the knowledge about single drugs to extrapolate the exposure to each of the APIs in a combinatorial treatment, as each API may interfere with the metabolism or action of the other(s). This can be even more sensitive when no improvement is expected or observed from one or more of the APIs of said combinatorial treatment, when used alone in the disease of interest.

The method applied by the inventors is a time and cost effective way to determine the best dose(s) of each API to be administered within a combinatorial treatment in order to obtain a more beneficial effect from said treatment. Though such a method can be used in all the different stages of drug development, it is of a particular interest in the frame clinical trials, where it allows a more rapid determination of the appropriate dose(s) and/or ratio(s) and/or dosage form(s) of the APIs to administer to the subject within said combinatorial treatment, which results in a significant gain in terms of time and cost. Indeed, a standard approach for a clinical trial with a combinatorial treatment would imply multiple dose studies for single drugs and combination thereof, besides placebo arms. This is of particular concern when single drugs (are expected to) have no or marginal effect on the disease to be treated when used alone. By allowing to diminish human testing, the method according to the invention also constitutes a significant advance in terms of ethics, particularly when considering life threatening diseases or serious diseases, particularly those for which no efficient treatment is currently available and consequently where administrating a placebo or an ineffective single API treatment could be considered as unethical.

Such a method is also an efficient way to determine whether a particular subject (or a subgroup of patients) is in need of a specific dosing of said combinatorial treatment, or of a specific ratio of the APIs that constitute said treatment, or a specific dosage form for one or more of said APIs, notably because of some peculiarities that alter PK/PD features of one or more of the APIs.

### Definitions

Within the context of the invention, the terms ‘combinatorial therapy’ relate to a combined use of at least two APIs in the frame of the treatment of a disease or improvement of a condition.

The positive enhancing effect of combined APIs can take several forms. When one API is active alone, the other(s) can potentiate said action through combined therapy. Another possibility is that, while each of the APIs when used alone shows weak to no efficacy, their combination results in an actual improvement of the condition or disease in the treated subject. Another form of enhancing effect of a combined therapy is when APIs simply cooperate to have an effect on a range of biological system or anatomical sites that are not completely covered by the APIs when used individually. In a particular embodiment, said APIs interact in a synergistic way. The term “synergy”, when applied to combinatorial treatments, means that said combinatorial treatment is at least significantly superior to the mere addition of the effects of each of the single APIs.

In a combinatorial therapy related to the invention, the APIs can be formulated together, or separately; when formulated separately, they can be administered simultaneously or sequentially.

Within the context of this invention, ‘PK parameters’ refers to pharmacokinetic parameters that are commonly used to describe the fate and the action of each API in a given body compartment when administered to an organism, in the present case a mammal. Particularly preferred PK parameters are the AUC₀₋ₜ, AUC_{0-inf}, and the Cₘₐₓ, which are well known from the skilled in pharmacology. Briefly, AUC means Area Under Curve and represents the bioavailability of the measured API. AUC₀₋ₜ is the time averaged concentration of the measured API observed from drug administration time (or just before administration, t=0) to a determinate time (t) whereas AUC₀₋ₜ (AUC to infinity) corresponds to the same parameter extrapolated from t=0 to infinity. Cₘₐₓ corresponds to the maximum concentration of the measured API that is reached in the tested body compartment. PK parameters can easily be measured in body fluids. Preferred body compartments to measure PK parameters are body fluids as blood, serum, plasma, cerebrospinal fluid and/or saliva.

“Outcomes” are defined as the occurrence or the strength of symptom(s), clinical sign(s) or laboratory abnormality(ies) in relation with a disease or with the administration of one or more of the API(s) of the combinatorial treatment. When related to the evaluation of the treatment of a disease they are categorized as “clinical endpoints”, “biomarkers” or “surrogate markers”. A ‘clinical endpoint’ is defined as a characteristic or variable that reflects how the patient feels or functions, or how long said patient survives. A ‘biomarker’ for a disease is a characteristic that is objectively measured and evaluated as an indicator of pathogenic process or pharmacologic response to a therapeutic intervention. A ‘surrogate biomarker’ is intended to substitute for a clinical endpoint and is expected to reasonably likely predict clinical benefit, harm or lack of benefit or lack of harm [3].

‘Laboratories abnormality’ refers to, for example, the differential level of concentration and/or the alteration of a nucleic acid, protein, metabolite or any molecule in the subject, when compared to a reference population or to a sample that is the signature of the presence, stage, worsening, improvement of the condition, disease or side effect which is studied.

Within the context of this invention, a ‘responder subject’ is a subject for who at least one endpoint, biomarker or surrogate marker in relation with the condition or disease to be treated is indicative of an actual effectiveness of the combinatorial treatment.

‘Subgroup of patients’ can correspond to patients that share common particularities that might influence on their physiological condition and/or metabolism. Said common particularities can be, for example, gender, ethnic group, body mass index, stage of the disease or condition that is studied, presence of an interfering condition or disease.

Within the context of the invention, the term ‘dosage form’ of the API(s) refers to any formulation of one or more of the API(s) that allows to reach particular pharmacokinetics profile(s), in order to obtain a more effective treatment. For example, it would be desirable to use a controlled release formulation for one or more of the API(s) of a combinatorial treatment in order to obtain a fully concerted action of the API(s), e.g. by, within a given body compartment, reaching the Cₘₐₓ of each of the API within the same time window from drug combination administration, or by maintaining a minimum concentration level of each API over a definite time window. The dosage form can be conditioned by the route of administration (e.g. topical, enteral or parenteral) which has been determined as the most suitable for obtaining a most effective treatment. It can also refer to modified drug release products. The conventional dosage forms generally allow an immediate release of the API(s), in some instances as explained above, a delayed release, an extended release, a targeted release, an orally disintegrating dosage form or even a microencapsulated form could be desirable in order to obtain a more effective treatment. Determining alternative dosage forms for a drug is a process well known from the skilled in the art, the pharmaceutical compositions may be formulated according to conventional pharmaceutical practice (see, e.g., Remington: The Science and Practice of Pharmacy (22nd ed.), eds. L.V. Jr., Allen A. Adej are, S. P. Desselle, L. A. Felton 2012 and Encyclopedia of Pharmaceutical Science and Technology, ed. J. Swarbrick (4^{th} edition) 2013, CRC press).

The method of the invention provides a new tool for aiding in the development of combinatorial therapies, more particularly determining more efficient and/or synergistic doses of each drug within said combinatorial therapy, taking into account the interrelationship of drugs within said combinatorial treatment and for which methods currently used for monotherapy are not suitable.

### Step i) of Selecting a Disease or Condition to be Treated

Methods of the invention are suitable for any disease or condition for which a combinatorial treatment is considered. Combinatorial treatments can represent a better strategy to obtain a better therapeutic efficacy, a decreased toxicity and/or reduced development of drug resistance compared to conventional monotherapies.

In a particular embodiment said methods are particularly useful for determining an optimal dose and/or dosage form and/or ratio of one or more of the APIs of a combinatorial treatment in the course of drug development program for multifactorial diseases or conditions or unmet medical needs for which a combinatorial therapy is contemplated.

In a particular embodiment the methods of the invention are applied in the context of the drug development programs for neurodegenerative disorders as Alzheimer's disease, Amyotrophic Lateral Sclerosis (ALS) and Parkinson's disease, diabetes, or peripheral neuropathies like, for example, Charcot-Marie-Tooth disease (CMT), or for improving conditions like e.g. memory performances.

Step ii) of Gathering, from Subjects Administered with one or more Doses or Particular Dosages of said Combinatorial Treatment, the Individual PK Parameters Corresponding to each API, or Metabolite(s) thereof.

PK parameters are used to determine the time course of the concentration of a drug in a body compartment, they reflect the exposure or, in relation with a chronic treatment, the impregnation of a subject with the treatment. PK parameters collection is routinely performed during drug experimentations, notably during clinical trials. They consist in the sum of the liberation, absorption, dispersion and metabolization of a medicine in an organism. The general health and/or way of life of said subject may alter such parameters from a subject to another. PK parameters of a drug also vary as a function of its mode and/or time of administration and of the potential interaction of other treatments with the metabolism of the drug that is studied. The collection of such parameters implies the identification and the quantification of either the drugs or the active moiety or derivative thereof that is responsible for its biological activity within a compartment (plasma, blood, serum etc. . . . ).

This degree of complexity is particularly increased in the case of combinatorial treatments, as one cannot rely on the knowledge about single drugs to extrapolate the exposure to each of the APIs when used in combinatorial treatment. Indeed, in the frame of a combinatorial therapy, APIs or metabolites thereof can interact and alter the fate of the other(s) once administered. Also the gathering of the PK data of the APIs in the course of the combinatorial treatment is essential.

Step iii) of Gathering from said Subjects the Data Corresponding to at Least one Clinical Endpoint, Biomarker and/or Surrogate Marker Related to the Selected Condition or Disease.

Outcomes to evaluate the symptomatic and/or actual correction or improvement of said disease are categorized as clinical endpoints, biomarkers or surrogate markers, as defined above.

In a particular embodiment said clinical endpoint(s), biomarker(s) or surrogate marker(s) can correspond to a detrimental side effect related to the use of one of the drugs of the combinatorial treatment or to the combinatorial treatment itself.

Outcome(s) can correspond to a particular single outcome or a group of outcomes related to one or several clinical sign(s) and/or symptom(s) and/or characteristic(s) of the disease. In a particular embodiment, said outcomes are grouped to constitute one or more composite outcome(s), said composite outcome(s) being indicative of the stage or the severity of the condition or disease, an improvement of said outcome(s) or composite outcome(s) therefore being the sign of a symptomatic and/or effective correction or improvement of said disease.

Outcomes can be of different nature. For example, they can be a (group of) physiological data, more particularly, current physiological parameters (as, for example, heart rate, cardiac interbeat interval, heart rate or heart rate variability, respiratory rate, thermal rate, blood volume pulse, or respiration rate . . . ) or electrophysiological parameters (e.g. EEG, sensitive or motor nerve conduction data). They can also be motor or cognition performance scores (e.g. speed of move, agility . . . ) or a statistically significant differential level in the concentration of a nucleic acid, protein, metabolite or any molecule or of an altered form thereof. Outcomes can also correspond to biochemical parameters determined from any type of sample from a subject (e.g. blood, urine biopsies, etc. . . . ). Of course, a composite outcome can groups the data related to outcomes of different nature.

During a clinical trial, outcomes are categorized as primary outcomes or secondary outcomes. A primary outcome is an outcome for which subjects have been randomized and for which the trial has been powered, whereas for a secondary outcome the trial may not have been powered nor randomized. Also, outcomes can be identified from a post hoc analysis of the data of a trial, i.e. they have not been specified a priori during the design of the study. Primary or secondary outcomes or outcomes resulting from a post hoc analysis of the data of the trial can be used to implement the method of the invention.

During pre-clinical studies, monotherapy with one or several of the APIS of the combinatory therapy might have been found ineffective in improving the relevant outcome(s), while the combinatorial therapy results in an actual improvement of the outcomes. The same is expected when moving to clinical trial. Consequently, in a particular embodiment, one or more API(s) of the combinatorial treatment, has or is expected to have, when administered alone, no effect on the measured clinical endpoint(s), biomarker(s) or surrogate marker(s).

Step iv) of Determining, for each of the APIs or Metabolite(s) thereof, the Clinical Endpoint(s), Biomarker(s) and/or Surrogate Marker(s) and the PK Parameter(s) for which a Correlation can be Established.

According to the method of the invention, once all the outcomes and PK data are gathered, they are analyzed in order to search for the PK data whose variation is linked to an alteration of an outcome.

It can be documented either by a mere linear correlation or a non-linear correlation.

In a particular embodiment, a positive correlation is observed between the PK data variation and the alteration of the outcome.

In another particular embodiment, an inverse correlation is observed between the PK data variation and the alteration of the outcome.

Statistical tests for determining said correlations are well known from the skilled in the art. As mentioned above said correlation can be found with one or several outcomes, with a subset of outcomes or with a composite outcome that is validated as a clinical endpoint, biomarker or a surrogate marker for the disease or condition to be treated.

Step v) of, Optionally, Selecting the most Relevant Clinical Endpoint(s), Biomarker(s) and/or Surrogate Marker(s) for the Selected Condition or Disease from those Determined in step iv).

In the case where alteration of several outcomes are found correlated with PK data of one or more API of the combinatory treatment, the data related to the most relevant outcome can be selected for the implementation of step vi). The most relevant outcome can be selected as a function of the general knowledge of the disease or condition. For example, it can correspond to the outcome that is the most commonly used in relation with said disease or condition.

Step vi) of Determining, from the Preceding step iv) or v), an Improved or Optimal or Selected Dose and/or Ratio and/or Dosage form of the API in the Context of the Combinatorial Therapy.

This step refers to the determination of a dose of each API for which a high effectiveness of combinatorial treatment is obtained or expected, with the fewest or without unacceptable side effect. More particularly, the invention takes advantage from the inter-individual variation in PK for the drugs. Indeed, inter-individual variations of PK parameters of a drug imply important variation of plasma concentration-time profiles after administration of the same dose of said drug to different subjects. This relies notably on a differential metabolization, excretion or biological availability from a subject to another. Such inter individual variation can be observed even within a group made of a small number of subjects and can cover a large interval of levels of drug impregnation. The individual effective body concentrations of drugs and their ratios determine different synergistic or additive actions of combinations. Thereby, comparing drug impregnation levels of subjects showing the best performances in outcome(s) correlated with an improvement or a correction of the condition or disease is indicative of whether an increase or a decrease (in comparison with the actual dosage administered to the patients) in the dosage of one or several of the API of the combinatorial treatment is desirable in order to obtain maximal effect and maximal positive pharmacodynamic interaction. This can also be indicative of the best ratio of APIs or of active metabolites thereof to reach in the body to obtain a more effective combinatorial treatment. This can indicate also the need of using particular dosage forms for one or more of the APIs in order to obtain a full concerted action of the drugs and thereby or more effective combinatorial treatment.

In a preferred embodiment, the selected dose and/or ratio and/or dosage form is a dose, ratio or dosage form of the API(s) for which the data related to the outcomes are indicative of an improvement or correction of the disease or condition to treat or of symptoms thereof.

In another preferred embodiment, the selected (e.g., improved or optimal) dose and/or ratio and/or dosage form is a dose, ratio or dosage form of the API(s) for which no or lower side effects are encountered by the subjects.

In an embodiment, the method of the invention comprises a step of identifying responder subjects within a group of subjects administered with said combinatorial treatment, wherein differential PK parameters in responder subjects are indicative of a need in increasing or diminishing the dosage, or modifying the dosage form, of at least one of the APIs for obtaining a more effective treatment in a largest population. As mentioned above, this method can be performed on a small group of subjects, thereby allowing to save time, costs and to spare in vivo testing in any stage of development of the combinatorial treatment. Said method is therefore of a particular interest during the dose finding phase of clinical trials. Consequently, in a more particular embodiment, the methods of the invention are applied to data gathered from human subjects.

In a particular embodiment, said method is performed with a group of 50, 40, 30, 20, or even less, subjects.

Though being particularly advantageous when used during clinical trials, this method is also of particular interest during the post marketing phase of a combinatorial treatment which, taking advantage from the enlargement of the treated population and the time of use of the medication, can uncover unexpected side effects in the target patient population, or in a subset thereof, or the identification of new or more efficient doses or ratios of the APIs within the combinatorial treatment. Consequently, in a particular embodiment, said method is performed at the later stages of clinical studies or during post marketing follow-up of the combinatorial treatment.

In a particular embodiment the PK parameters of the best responder subject(s) are significantly different from the median or mean value measured in the group of subjects administered with said combinatorial treatment.

In a more particular embodiment, the initial data on the relationship of PK parameters of individual APIs administered to individual patient with improvement of his/her symptoms or his/her secondary undesirable effects can be used together from larger population data in order to determine optimal doses of individual APIs from composition to achieve maximal therapeutic effect with minimal toxicity for this individual subj ect.

Said method can be used to adapt the combinatorial treatment to the particular need of a subject or of a particular group of subjects. Indeed, some subjects can present some specificities that alter the PK/PD of one or more drugs of the combinatorial therapy. Such an alteration may result in a less efficient therapy or even in an ineffective treatment or in unacceptable side effects. Such an alteration can be due, for example, to metabolic particularities of said subject or group of subjects or to treatments.

In a particular embodiment, determining the selected/optimal/improved dose and/or ratio and/or dosage form comprises comparing the selected PK parameters of an individual with a mean or median value determined in a group of subjects responding to said treatment and wherein a difference is indicative of a need in increasing or diminishing the dosage of at least one of the APIs within said combinatorial treatment. In a more particular embodiment, the individual (or a group thereof) in the above method presents metabolic or physiological particularities which can affect said ratio(s) or doses of APIs within the combinatorial therapy. Such peculiarity can be, for example, related to the age, weight, ethnic group, gender, or presence of other disease(s) or condition, or the taking of a treatment that interferes with metabolization of one or more of the APIs or affects the pharmacodynamics of the API(s) in said combinatorial treatment.

Of course, such increase or decrease should not lead to unacceptable side effects for the subject(s) to be treated.

Said method can be used to determine the doses of the single APIs or the ratios of doses that lead to a positive enhancing effect of combined action of the APIs that is expected in regard to a particular outcome. Indeed, it can be of particular interest for demonstrating superiority of combined therapy over single drug treatments, notably in the course of clinical trials. Consequently in a particular embodiment, the selected/optimal/improved dose and/or ratio and/or dosage is a dose and/or ratio and/or dosage form for which a positive enhancing effect of combined action of the APIs is expected. In a more particular embodiment, determining the doses and/or ratios and/or forms for which a positive enhancing effect of combined action of the APIs is expected, comprises building from step iv) or v) an exposure-effect relationship and determining from this relationship the doses and/or ratios and or dosage forms for which said enhancing effect is reached in the subjects.

As mentioned in the definition section, the synergistic effect of APIs within the combinatory treatment is defined as an effect that is significantly superior to the addition of the effects of each API. Consequently, in an even more particular embodiment, said positive enhancing effect of combined action of the APIs is synergy.

For a particular combinatorial treatment corresponding to specific values of PK parameters or ranges thereof for each of the APIs, synergy can be tested with an adaptation of effect-based strategy such as:
(i) Highest Single Agent [4] approach which reflects the fact that the resulting effect of a drug combination is greater than the highest effects produced by its individual components and, or
(ii) Response Additivity [5] approach which reflects that a positive drug combination effect occurs when the observed combination effect is greater than the expected additive effect given by the sum of the individual effects, and/or
(iii) Bliss independence model [6] which follows the principle that drug effects are outcomes of probabilistic processes and assumes that drugs act independently. It allows comparing the observed combination effect to the expected additive effect by the common formula for probabilistic independence. The model applies only to effects ranging between 0% and 100%.

Such methods allow avoiding the numerous testing groups that should be set up for the implementation of a factorial design of a clinical trial and the conventional demonstration of an actual superior effect of the combinatorial therapy. Such a method is thereby cost effective and allows to spare human tests that would be unethical. It is always advisable to dose a subject with the lowest dose of treatment that provides the best improvement of the disease or condition, which is difficult and time-consuming to determine using the usual “trial and error” model of dose finding process. In combinatory therapies, particularly low doses of each of the APIs are thought to be effective because of the positive enhancing effect of combined action of the APIs. The use such low doses is furthermore the best interest of treated subjects. The method of the invention is particularly suitable for determining more rapidly and accurately these lowest doses. Consequently in a particular embodiment, determining the doses and/or ratios for which a positive enhancing effect of combined action of the APIs is expected consists in searching for the lowest doses of the APIs within said combinatorial treatment. In a more particular embodiment, said doses represent 10% or less of the daily dose currently used for the API, when said API is an authorized medicine.

In a particular embodiment the methods of the invention comprise a further final step of administering the combinatorial treatment to the subject comprising the selected/optimal/improved dose, ratio and/or dosage form as determined in step vi) to a subject, or group thereof, in need to be treated for the selected disease or condition.

Further aspects and advantages of the invention shall be disclosed in the following experimental section, which is illustrative only.

### EXAMPLES

The study below was a double blind placebo control study performed in accordance with the European Medicines Agency ICH-E6 (R1) guideline recommendations and the French law n° 2004-806, Aug. 9, 2004 relative to public health law. The study was a preliminary study whose one of the objectives was to assess baclofen and acamprosate combinatorial treatment to improve cognition in young and elderly healthy volunteers. The study was conducted on a cohort of 24 human subjects.

The combination of baclofen and acamprosate has been found efficient in improving cognitive functions in treated healthy human subjects when compared to non-treated subjects. The question has been raised if the doses of individual drugs used in this clinical trial are optimal or whether a dose adjustment was necessary to expect a better response from subjects to the combinatorial treatment. An extensive post hoc analysis of the data collected from the treated subjects was performed.

1) Dosage Schedule

Briefly, the duration of treatment was of 10 days. Baclofen and acamprosate were given orally concomitantly as a combination therapy. After a scale up of doses during the first 4 days, maintenance regimen has been set at 15 mg baclofen and 1 mg acamprosate, or placebo, twice daily (morning and evening) till the day of testing (day 10).

2) PK Parameters

Plasma pharmacokinetics of baclofen and acamprosate have been established.
Cₘₐₓ, Tₘₐₓ, AUC₀₋ₜ have been determined for each of the drug given concomitantly as a combination therapy.
Cₘₐₓ is the observed maximum plasmatic concentration of acamprosate (or baclofen) measured in a subject at several time intervals after dosing.
Tₘₐₓ, is the time at which Cₘₐₓ was apparent, identified by inspection of the plasma drug concentration vs. time.
AUC₀₋ₜ is the area under the concentration-time curve from time zero (pre-dose) to the time of last quantifiable concentration, it was calculated using a linear trapezoidal method.

Baclofen and acamprosate concentrations have been determined at 0.5, 1, 1.5, 3, 5, 8 and 24 h post-dose in plasma samples. Blood was collected via venipuncture or cannulation of a forearm vein(s). Plasma was immediately separated in a refrigerated centrifuge (ca. +4° C.) at 1600 g for 10 min and the resulting plasma was analyzed for acamprosate and baclofen using a validated LC-MS/MS method. The PK analyses were carried out by using WinNonlin Professional software (Version 5.3 or higher—Pharsight Corporation—Mountain View, California—USA). A Non Compartmental approach was chosen.

Tₘₐₓ has been determined as occurring at 1.5-2 hours after the administration of the combinatorial treatment for acamprosate and at 1.5 hours for baclofen.

3) Endpoint Measurements

Several tests were performed to measure different outcomes related to cognitive performances of the subjects, notably Cogstate® cognitive tests and cognitive Event-Related Potentials (ERPs) were assayed.

Electrophysiological measurement of Cognitive Event-Related Potentials (ERPs) were recorded to assess the cognitive performances of the subjects. ERPs are the electrophysiological response to a stimulus and could serve as a surrogate biological marker of cognitive performances [7]. Here, the cognitive task required paying attention and to count odd stimuli according to a specific protocol. Potential is recorded during the test. An ERP latency composite score is determined from the different derivations. A decrease of latency (i.e. an increase of composite score) of P300 wave and its subcomponents in response to odd stimuli is believed to be the sign of an improvement of electrophysiological processes underlying cognitive performances of the subjects.

These tests were performed all through the study; results at Day 10 (at 6 hours post-dose) were studied in relation with the PK data of the patients determined for baclofen and acamprosate (measured at 0.5; 1; 1.5; 3; 5; 8 and 24 h post-dose). A correlation was searched between each of the different endpoints (i.e. the cognitive tests, or the ERP latency composite score) and drug concentrations determined at each sampling time.

4) Data Statistical Analysis

### Correlation Study

Correlation between PK variables and endpoints results has been systematically sought.

Pearson's correlation coefficient was determined for each of the endpoints and different plasmatic concentrations measured as a function of time. Significance of the correlation was tested using a t-test with HO being r=0 and H1 r≠0, to determine whether the slope is significantly different from 0.

### 3D Representation

Statistical analysis was performed with R (R Core Team (2015). R: A language and environment for statistical computing. R Foundation for Statistical Computing, Vienna, Austria. URL: https ://www.R-project.org/.).

Given magnitude difference between acamprosate and baclofen concentrations, data were normalized between 0 and 1. For each drug, 0 corresponded to the min concentration and 1 to the max concentration and all other values were transformed to be between 0 and 1.

The variability in both drug AUC and ERP composite score and the link between them can be used to build an AUC-ERP composite score relationship that can be displayed in a 3D graphic in order to extrapolate linearly the AUC-ERP composite score-surface.

5) Results

Improvement of Memory and Memory Related Mental Functions is Linearly Correlated with Plasmatic Concentrations of Drugs.

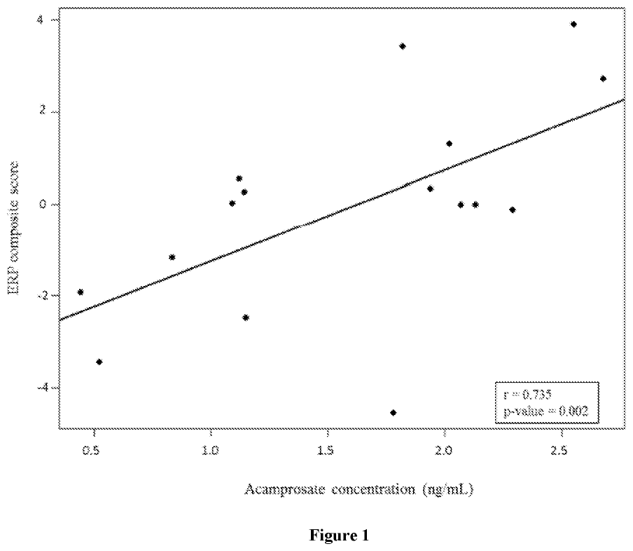

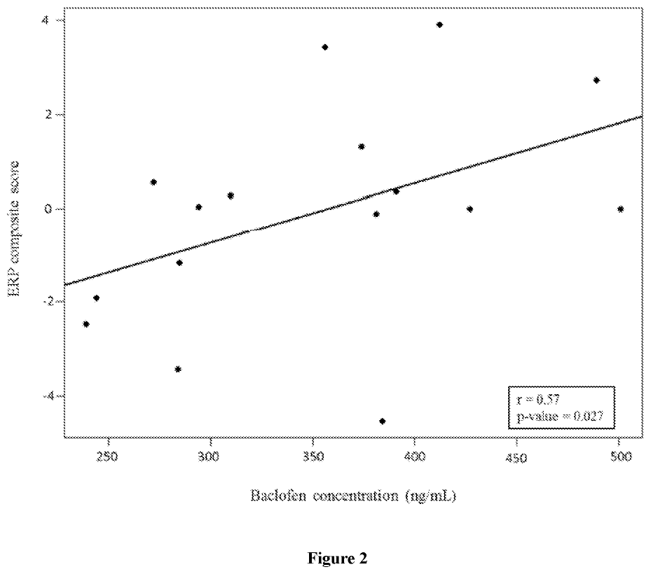

FIGS. 1 and 2 clearly illustrate the inter-individual variation of drug plasmatic concentration. This variation allowed determining whether an increase or a decrease in the dosing of one or the two drugs is desirable to expect an improvement of combinatorial treatment with baclofen and acamprosate.

As shown in FIGS. 1 and 2 a significant correlation was found between plasma concentration of the drugs and ERP composite scores of the subjects.

When looking at electrophysiological component of memory and related mental functions, a significant correlation is observed between the plasmatic concentrations of acamprosate and baclofen, 1.5 hours after administration, with the respective ERP latencies composite score (measured 6 hours after the administration of the drugs). As shown in FIGS. 1 and 2, plasmatic concentrations of both drugs are positively correlated with latencies in ERPs. Subjects performing the best (i.e. with the shorter latencies) are those for which the higher plasmatic concentration is observed at 1.5 hour. Noticeably, as stated above, 1.5 hour from drug administration corresponds to the Tₘₐₓ of baclofen and roughly to the Tₘₐₓ of acamprosate.

No correlation was found for the other measured PK parameters.

Clearly, this data analysis shows that individuals exhibiting higher baclofen and acamprosate plasmatic concentration perform better. Of note, these concentrations are positively correlated with ERP composite scores, and quite different from the mean concentration observed in the whole sample of treated individuals (table 1). Consequently a significant improvement of cognition can be expected from an increase in the dosing of baclofen (15 mg bid in the trial), acamprosate (1 mg bid in the trial) or both; said increase should be contemplated taking into account known potential side effects attached to higher doses of the single drugs.

TABLE 1

| | | Mean | Most efficient | |
|---|---|---|---|---|
| | | plasmatic | plasmatic | Correlation |
| | | concentration | concentration | coefficient |
| Endpoint | | (ng/ml) | in trial (ng/ml) | (p-value) |
| ERPs | baclofen^{¥} | 350.6 | ≥500 | 0.570 (0.027) |
| latencies | acamprosate^{¥} | 1.59 | >2.5 | 0.735 (0.002) |
| composite | | | | |
| score | | | | |

^{¥}Drug concentration at Tₘₐₓ (i.e. 1.5 h from drug administration) are found correlated with ERP latencies composite score

Determining Ranges of Ratios for which an Improvement in Cognitive from the Individual Pharmacological Data.

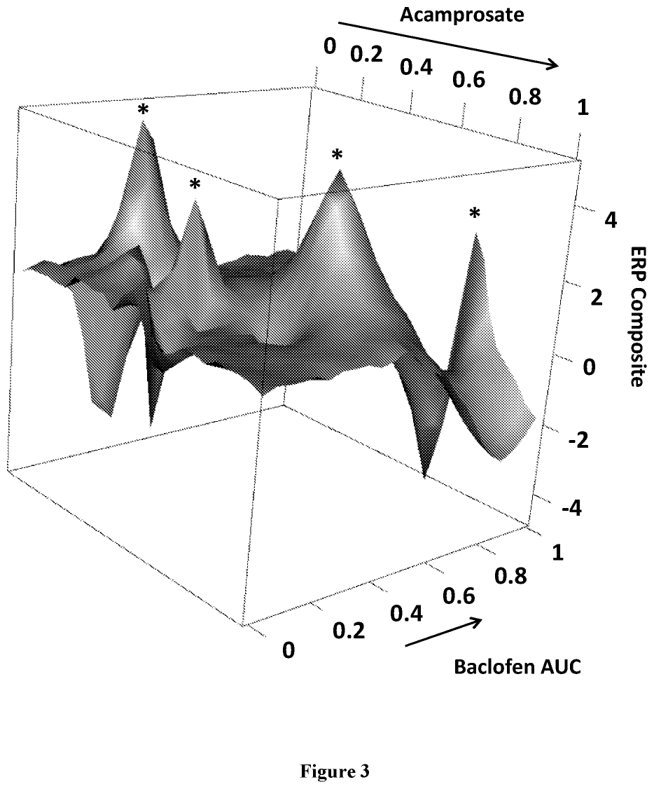

Determining the most effective ranges of ratio of doses of drugs within a combinatorial treatment is of an outmost importance. Indeed, improving a multifactorial disease implies the fine tuning of different targets or pathways though specific ranges of ratios for the drugs acting on these targets and/or pathways. FIG. 3 illustrates that it is possible to determine, from the inter-individual variation of drug plasmatic concentration, the (ranges of) ratios for which an improved efficacy can be expected. The plotting of the respective composite score of ERP latencies of each subject of the study with drug plasma concentrations allows delineating a surface representing the efficacy of the combined therapy as a function of drug concentration.

Peaks (FIG. 3) delineate ranges of drug concentrations for which the subjects perform the better, whereas valleys mark the ranges of drugs concentrations for which subjects perform the worst. Hence from these figures, one can easily determine the ranges of ratios of baclofen and acamprosate plasmatic concentrations at which an improvement of cognitive performances should be observed. The actual dosage of drug within the combinatorial therapy can easily be deduced from these concentrations.

### BIBLIOGRAPHY

1 Chumakov I, Nabirotchkin S, Cholet N, Milet A, Boucard A, Toulorge D, Pereira Y, Graudens E, Traoré S, Foucquier J, Guedj M, Vial E, Callizot N, Steinschneider R, Maurice T, Bertrand V, Scart-Gres C, Hajj R & Cohen D (2015) Combining two repurposed drugs as a promising approach for Alzheimer's disease therapy. Sci. Rep. 5, 7608.
2 Chumakov I, Milet A, Cholet N, Primas G, Boucard A, Pereira Y, Graudens E, Mandel J, Laffaire J, Foucquier J, Glibert F, Bertrand V, Nave K-A, Sereda MW, Vial E, Guedj M, Hajj R, Nabirotchkin S & Cohen D (2014) Polytherapy with a combination of three repurposed drugs (PXT3003) down-regulates Pmp22 over-expression and improves myelination, axonal and functional parameters in models of CMT1A neuropathy. Orphanet J. Rare Dis. 9, 201.
3 De Gruttola VG, Clax P, DeMets DL, Downing GJ, Ellenberg SS, Friedman L, Gail MH, Prentice R, Wittes J & Zeger SL (2001) Considerations in the evaluation of surrogate endpoints in clinical trials. summary of a National Institutes of Health workshop. Control. Clin. Trials 22, 485-502.
4 Geary N (2013) Understanding synergy. Am. J. Physiol. Endocrinol. Metab. 304, E237-53.
5 Slinker BK (1998) The statistics of synergism. J. Mol. Cell. Cardiol. 30, 723-31.
6 Bliss CI (1939) The toxicity of poisons applied jointly. Ann. Appl. Biol. 26, 585-615.
7 Bennys K, Portet F, Touchon J & Rondouin G (2007) Diagnostic value of event-related evoked potentials N200 and P300 subcomponents in early diagnosis of Alzheimer's disease and mild cognitive impairment. J. Clin. Neurophysiol. 24, 405-12.

1. A method for determining an optimal dose and/or dosage form and/or ratio for one or more active pharmaceutical ingredients (APIs) of a combinatorial treatment for a selected condition or a disease, said method comprising:
i. selecting a condition or a disease to be treated,
ii. gathering from subjects administered with one or more doses or dosage forms of said combinatorial treatment, individual pharmacokinetics (“PK”) parameters corresponding to each API or metabolite(s) thereof,
iii. gathering from said subjects data corresponding to at least one clinical endpoint, biomarker and/or surrogate marker related to the selected condition or disease,
iv. determining, for at least one API or metabolite thereof, the clinical endpoint(s), biomarker(s) and/or surrogate marker(s) and the PK parameter(s) for which a correlation can be established,
v. optionally, selecting the most relevant clinical endpoint(s), biomarker(s) and/or surrogate marker(s) for the selected condition or disease from those determined in step iv), and
vi. determining, from the preceding step iv) or v), an optimal dose and/or dosage form and/or ratio of one or more of the APIs to be administered in said combinatorial treatment to obtain a more effective treatment of said condition or disease.

2. The method according to claim 1, wherein the PK parameter(s) are selected from the maximum concentration of the measured API(s) or of metabolites thereof reached in a body compartment (Cₘₐₓ) and/or the bioavailability of the measured API(s) or of metabolites thereof (AUC₀₋ₜ).

3. The method according to claim 1, wherein the PK parameters of the APIs or of metabolite(s) thereof are determined from a blood, plasmatic or sera sample(s) of the subjects.

4. The method according to claim 1, wherein said combinatorial treatment comprises 2 or 3 APIs.

5. The method according to claim 1, wherein determining an optimal dose and/or ratio and/or dosage form of one or more of the APIs of step vi) comprises determining the doses and/or ratios and/or dosage form leading to a positive enhancing effect of combined action of the APIs.

6. The method according to claim 5, comprising building from step iv) or v) an exposure-effect relationship and determining from this relationship the doses and/or ratios and/or dosage form for which said positive enhancing effect of combined action is reached in the subjects.

7. The method according to claim 5, wherein said positive enhancing effect is synergy.

8. The method according to claim 1, wherein determining an optimal dose and/or ratio and/or dosage form of one or more of the APIs of step vi) comprises comparing the selected PK parameters of a subject with a mean or median value observed in a group of subjects responder to said treatment and wherein a difference is indicative of a need in increasing or diminishing the dose of at least one of the APIs within said combinatorial treatment.

9. The method according to claim 8 wherein said subject is subjected to a medication that might interfere with the metabolism of one or more of the APIs of the combinatorial treatment.

10. The method according to claim 1, wherein determining an optimal dose and/or dosage form and/or ratio of the APIs of step vi) comprises identifying responder subjects within a group of subjects administered with said combinatorial treatment, wherein differential PK parameters in responder subjects are indicative of a need in increasing or modifying ratio or diminishing the dosage, or the dosage form, of at least one of the APIs for obtaining a more effective treatment in a largest population.

11. The method according to claim 1, wherein a more effective treatment is a treatment for which less or no side effect in relation with the use of one or more of the APIs is noticed in the subject.

12. The method according to claim 1, wherein the subject(s) is (are) human subject(s).

13. A method for optimizing therapeutic efficiency of a combinatorial treatment of a selected condition or a disease, said method comprising:
comparing individual PK parameters for each API of said combinatorial treatment in subjects previously treated with one or several doses or dosage forms of the APIs in the combinatorial treatment,
determining a PK data variation, and
correlating said PK data variation with an alteration of clinical outcome of the selected disease,
said correlation allowing to determine an optimal dose or dosage form of each API for said combinatorial treatment, leading to an optimization of therapeutic efficiency.

14. A method for treating a subject having a disease with a combinatorial treatment of active pharmaceutical ingredients (APIs), said method comprising (i) determining an optimal dose or dosage form or ratio of each API for said combinatorial treatment of said disease according to claim 1 and (ii) treating the subject with said optimal dose or dosage form or ratio of each API.
